(19) United States
(12) Patent Application Publication
(10) Pub. No.: US 2010/0047210 A1
(43) Pub. Date: Feb. 25, 2010

(54) Systems and Methods for Positioning of Needles and Other Devices Within Body Tissue

(75) Inventors: Jeremy Johnson, San Francisco, CA (US); Aine Smalley, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/197,570

(22) Filed: Aug. 25, 2008

(51) Int. Cl. A61K 35/12; A61K 38/02; A61K 31/70; A61K 33/00; A61P 9/00; A61K 38/16; A61K 38/14

(52) U.S. Cl. 424/93.7; 514/2; 514/23; 424/718; 514/12; 514/8; 424/93.1; 604/103.03

(57) ABSTRACT

Systems and methods for delivering a substance (e.g., a therapeutic or diagnostic substance, drug, biologic, cells, etc.) or device (e.g., implantable device, sensor, electrode, etc.) to a specific location within a tissue mass (e.g., an organ, tumor, other mass of body tissue, etc.) that is located a spaced distance from an open or fluid-filled space, body cavity or body lumen.

### FIELD OF THE INVENTION

The present invention relates generally to methods and apparatus for medical treatment and more particularly to systems and methods for controlling the position of a needle or other device within the wall of an organ or other body tissue mass.

### BACKGROUND

In modern medicine there are numerous situations in which it is desirable to control the depth of penetration or positioning of a needle or other therapy delivering or diagnostic device that has been advanced into the wall of an organ, tumor or other body tissue mass. In some cases, the needle or other therapy delivering or diagnostic device is advanced into the desired anatomical structure by hand. In other cases, the needle or other therapy delivering or diagnostic device is advanced from a catheter that has been inserted into the patient's vasculature. Examples of catheters that have needles or other treatment devices that may be advanced from the catheter into adjacent tissue are described in U.S. Pat. No. 5,830,222 (Makower); U.S. Pat. No. 6,068,638 (Makower), U.S. Pat. No. 6,159,225 (Makower), U.S. Pat. No. 6,190,353 (Makower, et al.), U.S. Pat. No. 6,283,951 (Flaherty, et al.), U.S. Pat. No. 6,375,615 (Flaherty, et al.), U.S. Pat. No. 6,508,824 (Flaherty, et al.), U.S. Pat. No. 6,544,230 (Flaherty, et al.), U.S. Pat. No. 6,655,386 (Makower et al.), U.S. Pat. No. 6,579,311 (Makower), U.S. Pat. No. 6,602,241 (Makower, et al.), U.S. Pat. No. 6,655,386 (Makower, et al.), U.S. Pat. No. 6,660,024 (Flaherty, et al.), U.S. Pat. No. 6,685,648 (Flaherty, et al.), U.S. Pat. No. 6,709,444 (Makower), U.S. Pat. No. 6,726,677 (Flaherty, et al.) and U.S. Pat. No. 6,746,464 (Makower) and co-pending U.S. patent applications having Ser. Nos. 11/279,993; 11/279,265; 11/279,771; 11/610,092; 11/534,895; 11/613,764; 11/837,718; 12/054,533 and 12/045,120, the entire disclosures of which are expressly incorporated herein by reference.

In particular, it is sometimes desirable to control the depth at which a therapeutic substance is injected into an organ, tumor or other anatomical structure. For example, various therapeutic substances may be injected into ischemic, infracted or damaged areas of a patient's heart in order to stimulate neoangiogenesis in ischemic myocardium, establish new functional cardiac muscle cells, deter ventricular remodeling, deter scar formation at an infarct site or otherwise improve/maintain myocardial function. In some instances, these therapeutic substances may be combined with other ancillary agents (e.g., potentiators or radiographic markers) when injected into the myocardium.

The types of angiogenic substances that have been proposed for injection into the myocardium to stimulate neoangiogenesis in ischemic myocardial tissue include peptide growth factors, small molecule drugs and other active compounds, biologically active carbohydrates, recombinant biopharmaceuticals, agents that are active in the regulation of vascular physiology and cellular and gene therapy agents. Examples of specific angiogenic substances that fall within these general categories include but are not limited to: nitric oxide, agents that affect transcription or turnover of cellular mRNA or the efficiency with which specific mRNA translates into its protein product, antisense agents, hormones, soluble receptors, receptor ligands, synthetic and naturally occurring peptides, peptidomimetic compounds, specific and non-specific protease inhibitors, postaglandins, inhibitors of prostaglandin synthase and/or other enzymes involved in the regulation of prostaglandin synthesis, fibroblast growth factors (FGF's), acidic (aFGF, FGF-II) and basic (bFGF, FGF-I) fibroblast growth factors, vascular endothelial growth factors (VEGF), agents that stimulate endogenous production of VEGF such as platelet-derived growth factor (PDGF), purified monocyte-derived angiogenic substance (angiotropin), angiogenin, transforming growth factor alpha (TGF-α), transforming growth factor beta (TGF-β) and angiogenic cell precursors (e.g., stem cells).

Examples cell preparations that are purported to be injectable into the myocardium for the purpose of establishing new functional heart muscle include myocyte preparations, embryonic, adult and mesenchymal stem cell preparations, myoblast (including skeletal myoblast) preparations, bone marrow mononuclear cell preparations and possibly others.

Also, a substance that has been found to deter deleterious remodeling of the ventricle wall or otherwise improve myocardial function when injected to an area of infracted myocardium is platelet gel (PG). In some instances, platelet gel components (e.g., platelet concentrate and a thrombin containing solution) may be injected through separate lumens so that they become combined to form platelet gel immediately before or after entering the myocardium, as described in United States Patent Application Publication Nos. 2006/0041243 (Ser. No. 11/159,752) entitled Devices And Methods For Interstitial Injection Of Biologic Agents Into Tissue; 2007/0014784; 11/426219 (Ser. No. 11/426,219) entitled Methods and Systems for Treating Injured Cardiac Tissue; 2007/0042016 (Ser. No. 11/426,211) entitled Methods and Systems for Treating Injured Cardiac Tissue; 2007/0093748 (Ser. No. 11/643,359) entitled Methods and Systems for Treating Injured Cardiac Tissue; 2007/0172472 (Ser. No. 11/619,576) entitled Methods and Systems for Treating Injured Cardiac Tissue; 2008/0161757 (Ser. No. 11/969,085) entitled Devices and Methods for Injection of Multiple-Component Therapies 2008/0161772 (Ser. No. 11/969,094) entitled and Devices and Methods for Injection of Multiple-Component Therapies as well as copending U.S. patent application Ser. No. 12/106,839 entitled Dual Syringe Injector System, the entire disclosure of each such patent application being expressly incorporated herein by reference.

Also, it is sometimes desirable to deliver various other diagnostic, cosmetic or therapeutic substances (drugs, chemotherapeutic agents, other anti-cancer agents, radiographic contrast materials, biologics, cell preparations, gene therapy preparations, bulking agents, fillers, etc.) or to deliver implantable devices (e.g., drug delivery implants, electrodes, radiographic markers or tags, nanotechnology devices, etc.) through needles, cannulae or other elongate devices that have been inserted or advanced to specific locations within organs or tissue masses (normal or aberrant) within the bodies of human or animal subject. In many such cases, controlling the depth at which the substance or device is delivered into tissue is desirable.

The prior art has included a number of devices for limiting or controlling the depth to which a needle, injector or other device penetrates into a tissue mass when inserted by hand or from a catheter or other device. For example, U.S. Pat. No. 3,538,916 describes an injection pistol for intramuscular implantation of encapsulated liquid or solid chemical material. The depth of injection of the needle is controlled by an injection depth gauge mounted on the injection needle. A shaft having a slidable plunger integral therewith is mounted on the frame and is utilized to eject the material from the needle after the needle has been advanced into the muscle. The travel of the plunger within the injection needle is limited by an adjustable depth stop mounted on the end of the shaft opposite the plunger.

U.S. Pat. No. 4,270,537 (Romaine) describes a hypodermic syringe and automatic needle insertion device wherein the syringe is biased against a trigger when the needle is in the retracted position. Upon release of the trigger, the syringe and needle are driven forward extending the needle into the underlying tissue. The depth of insertion may be predetermined by the attachment of an interchangeable stop.

U.S. Pat. No. 4,710,171 (Rosenberg) describes a needle depth setting sheath assembly and needle stop which comprises a locking assembly that may be tightened about a “needle barrel.” A sheath with graduations therein permits the needle depth to be adjusted accurately. An elastomeric bushing is compressible about the “needle barrel,” and the bushing is prevented by a screw-hub arrangement from becoming dislocated.

U.S. Pat. No. 5,141,496 (Dalto, et al.) describes a syringe guide with adjustment of the depth to which the needle penetrates. One end of the syringe guide has a sliding base which is adjustable by means of a screw and the other end includes a spring-loaded sliding portion that is affixed to the syringe and propels the needle to a predetermined depth of injection.

U.S. Pat. No. 5,217,438 (Davis et al.) describes a needle stop for a biopsy needle or the like wherein a coil spring is engaged with the surface of the shaft of the needle. Ends of the spring may be pinched to enlarge the spring coils and permit movement of the stop along the shaft. A guard tube is associated with the stop for covering the point of the needle when not in use.

U.S. Pat. No. 5,250,026 (Ehrlich et al.) describes an implant injector that has an adjustable insertion depth feature. The insertion depth adjusted by moving the nose of the injector relative to the tip of the cannula that extends past the nose. In addition to adjusting the insertion depth, the cannula or needle, may also be rotated to a plurality of positions relative to the injector handle. A spring loaded plunger, when released by a release button, will push the implant out the end of the cannula as the operator withdraws the cannula from the animal. The release button is designed as a safety trigger to avoid premature activation of the plunger during insertion of the needle. Needles, or cannulae of various diameters and lengths, may be interchanged in the injector. Also, the spring loaded plunger for expelling the implant may be removed allowing the operator to replace the plunger with a different diameter and length plunger, if desired, to match different size cannulae.

U.S. Pat. No. 6,623,474 (Ponzi) describes a injection catheter that is equipped with a needle stop. The catheter comprises a catheter body comprising a flexible tubing having proximal and distal ends and at least one lumen therethrough. A tip section comprising a flexible tubing is mounted at the distal end of the catheter body. The tip section has a needle passage extending therethough. The needle passage has a proximal region having a proximal diameter and a distal region having a distal diameter less than the proximal diameter. A needle control handle is provided at the proximal end of the catheter body. An injection needle extends through the tip section, catheter body, and needle control handle and has a proximal end attached to the needle control handle and a distal end within the needle passage. The injection needle is longitudinally slidable so that its distal end can extend beyond the distal end of the tip section upon suitable manipulation of the needle control handle.

U.S. Pat. No. 5,102,393 (Sarnoff et al.) describes an autoinjector that has an intramuscular injection mode and a subcutaneous injection mode. An injection mode converting structure is useable to convert the device back and forth between a subcutaneous mode wherein the needle is allows to advance to a first depth that does not extend substantially beyond subcutaneous tissue at the injection site and an intramuscular mode wherein the needle is allowed to advance to a second depth that is within muscle that underlies the subcutaneous tissue.

Also, copending U.S. patent application Ser. No. 12/108,961 (Macaulay et al.) describes devices and methods for limiting the depth to which a penetrator is advanced into an organ or mass of tissue. This application describes a device that generally comprises a first member and a second member, with a penetrator attached to and extending from the second member. The first member has a penetrator shroud and a hollow bore extending therethrough. The second member is engageable with the first member such that a distal portion of the penetrator extends through the penetrator shroud. The distance to which the penetrator protrudes out of and beyond the distal end of the penetrator shroud is adjustable in accordance with the desired depth of penetration. The penetrator may then be advanced into the organ or tissue mass until the distal end of the shroud abuts against the organ or tissue mass, thereby stopping further advancement of the penetrator. The penetrator may have one or more lumen(s) for aspirating or infusing substances.

There remains a need in the art for the development of additional devices and methods for controlling the depth or positioning of needles, cannulae and other diagnostic/therapeutic devices within the walls of organs or other tissue masses.

### SUMMARY OF THE INVENTION

In accordance with one aspect of the present invention, there is provided a system for positioning a treatment delivery location (e.g., on opening, port, lens or other location from which a therapeutic or diagnostic substance, flow of energy or device may be delivered) of an elongate treatment device at a desired location within a tissue mass located near an open or fluid-filled space, body cavity or body lumen, said system. In general, this system comprises (A) an elongate treatment device having a treatment delivery location that is advanceable into the tissue mass; (B) an anchoring member comprising a shaft and an anchor, said anchor having a radially collapsed configuration and a radially expanded configuration, said shaft being advanceable into the space, body cavity or body lumen while the anchor is in its collapsed configuration, the anchor being thereafter transitionable to an expanded configuration and the shaft being thereafter retractable to pull the anchor into abutment with a wall of the space, body cavity or body lumen adjacent to the tissue mass and (C) measuring apparatus useable to determine the distance between the location at which the anchor abuts against the wall of the space, body cavity or body lumen and a treatment-delivering location on the elongate treatment device.

Further in accordance with the present invention, there is provided a method for positioning a treatment delivery location of a treatment device within a tissue mass at a desired distance from an open or fluid-filled space, body cavity or body lumen. In general, this method comprises the steps of (A) advancing an anchoring member from the treatment device and into the space, body cavity or body lumen, (B) causing an expandable anchor on the anchoring member to expand, (C) retracting the anchoring member to cause the expanded anchor to abut against a wall of the space, body cavity or body lumen, (D) measuring the distance between the treatment delivery location on the elongate treatment device and the location at which the anchor abuts against the wall of the space, body cavity or body lumen and (E) maneuvering the elongate treatment device as needed until the measured distance indicates that the treatment delivery location of the elongate treatment device is positioned at the desired distance from the space, body cavity or body lumen.

Further aspects, details and embodiments of the present invention will be understood by those of skill in the art upon reading the following detailed description of the invention and the accompanying drawings.

### BRIEF DESCRIPTION OF THE DRAWINGS

FIG. 1 is a side perspective view of one embodiment of an injector system of the present invention which generally includes a dual lumen injector cannula and an inflatable anchoring member.

FIG. 1A is a transverse cross sectional view through line 1A-1A of FIG. 1.

FIG. 2 is a side view of the anchoring member of the system shown in FIG. 1.

FIGS. 3A-3G show steps in a method for using the injector system of FIG. 1 to inject a diagnostic or therapeutic substance at a desired location within a tissue mass located adjacent to an open, hollow or fluid-filled space.

FIG. 4 is a side perspective view of another embodiment of an injector system of the present invention which generally includes a dual lumen injector cannula and a coiling anchoring member.

FIG. 4A is a transverse cross sectional view through line 4A-4A of FIG. 4.

FIGS. 5A-5G show steps in a method for using the injector system of FIG. 4 to inject a diagnostic or therapeutic substance at a desired location within a tissue mass located adjacent to an open, hollow or fluid-filled space.

FIG. 6 is a side perspective view of another embodiment of an injector system of the present invention which generally includes a single lumen injector cannula and a coiling anchoring member.

FIG. 6A is a transverse cross sectional view through line 6A-6A of FIG. 6.

FIGS. 7A-7G show steps in a method for using the injector system of FIG. 6 to inject a diagnostic or therapeutic substance at a desired location within a tissue mass located adjacent to an open, hollow or fluid-filled space.

### DETAILED DESCRIPTION

The following detailed description and the accompanying drawings are intended to describe some, but not necessarily all, examples or embodiments of the invention. The contents of this detailed description and accompanying drawings do not limit the scope of the invention in any way.

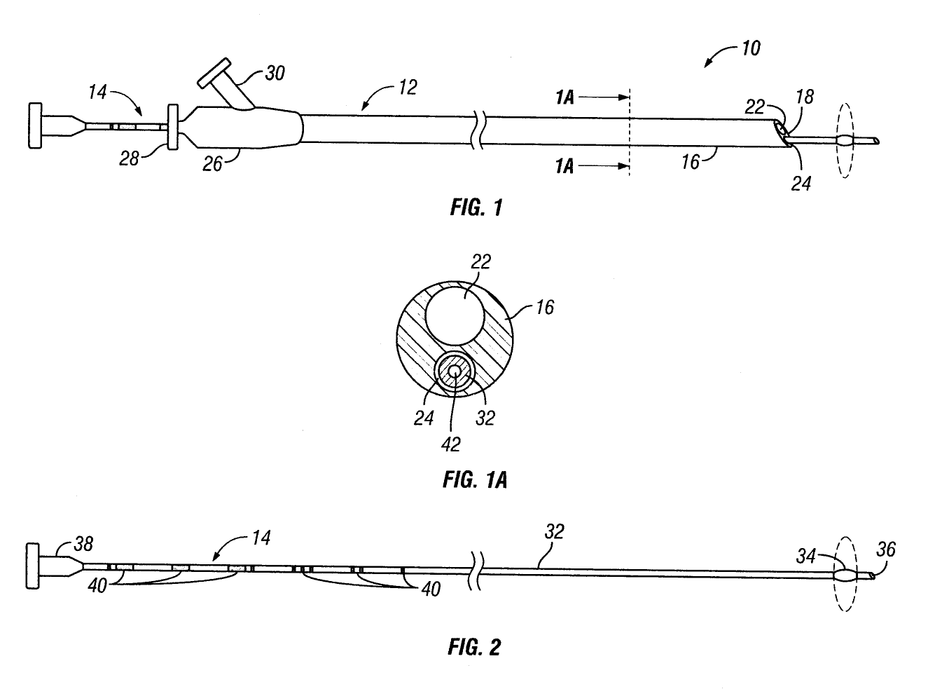

FIGS. 1, 1A and 2 show one particular embodiment of an injector system 10 of the present invention. This injector system 10 generally comprises an elongate treatment device in the nature of an injector cannula (e.g., a needle) 12 and an anchoring member 14. In this non-limiting example, the injector cannula 12 comprises an elongate shaft 16 having a tissue penetrating distal end 18 and at least two lumens extending longitudinally therethrough, namely an infusion lumen 22 and an anchoring member lumen 24. Additional lumens may also be provided in some variations of this device 12. A proximal hub 26 is attached to the proximal end of the elongate shaft 16. The proximal hub 26 has an anchoring member port in the nature of a proximal Luer port 28 which communicates with the anchoring member lumen 24 and an infusion port which, in this example, comprises a side Luer port 30 which communicates with the infusion lumen 22. Also, in this example, the anchoring member 14 comprises an elongate shaft 32 having a tissue-penetrating distal end 36, a straight Luer hub 38 on its proximal end, a balloon 34 located on a distal portion of the shaft 32 and graduated distance markers 40 on a proximal portion of the shaft 32.

As may be appreciated from the showings of FIGS. 1 and 1A, the anchoring member shaft 32 is inserted through the proximal Luer port 28 and is advanceable through the anchoring member lumen 24. The graduated distance markers 40 on the proximal portion of the shaft 32 are located at spaced-apart increments so that, as the distal end 36 of the anchoring member shaft 32 is advanced out of the distal end 18 of the injection cannula 12, the markers 40 will provide to the operator an indication of the current distance between the balloon 34 (when inflated) and the distal opening of the infusion lumen 22. For example, in the embodiment shown in the drawings, the distal opening of the infusion lumen 22 is through the distal end 18 of the injection cannula 12 and the markers 40 may be spaced apart in 5 millimeter increments so as to signify the following positions as the anchoring member 14 is advanced:
Position 1. When the first (i.e., distal-most) marker 40 is flush with the proximal end of the hub 26, the distal end 36 of the anchoring member 14 is flush with the distal end 18 of the injector cannula 12.
Position 2. When the second marker 40 is flush with the proximal end of the hub 26, the proximal end of the balloon 34 (when deflated) is flush with the distal end 18 of the injector cannula 12.
Position 3. When the third marker 40 is flush with the proximal end of the hub 26, the distance between the distal opening of the infusion lumen 22 and the proximal side of the balloon 34 (when inflated) is 5 millimeters.
Position 4. When the fourth marker 40 is flush with the proximal end of the hub 26, the distance between the distal opening of the infusion lumen 22 and the proximal side of the balloon 34 (when inflated) is 10 millimeters.
Position 5. When the fifth marker 40 is flush with the proximal end of the hub 26, the distance between the distal opening of the infusion lumen 22 and the proximal side of the balloon 34 (when inflated) is 15 millimeters.
Position 6. When the sixth marker 40 is flush with the proximal end of the hub 26, the distance between the distal opening of the infusion lumen 22 and the proximal side of the balloon 34 (when inflated) is 20 millimeters.
Position 7. When the sixth marker 40 is flush with the proximal end of the hub 26, the distance between the distal opening of the infusion lumen 22 and the proximal side of the balloon 34 (when inflated) is 20 millimeters.

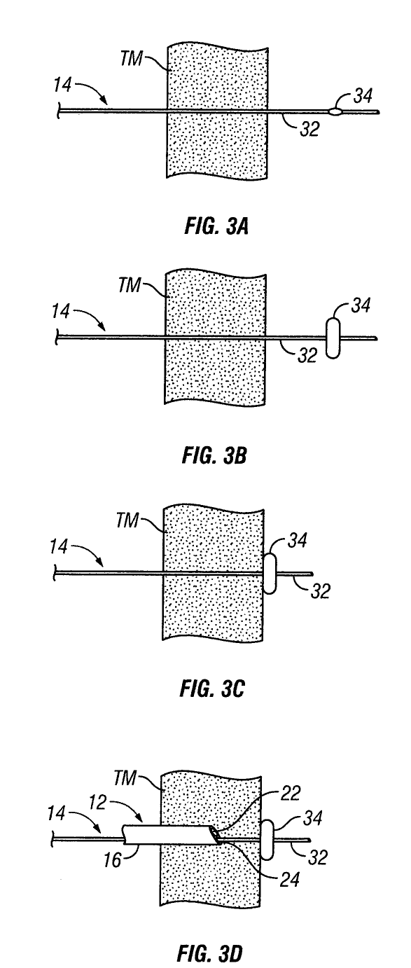

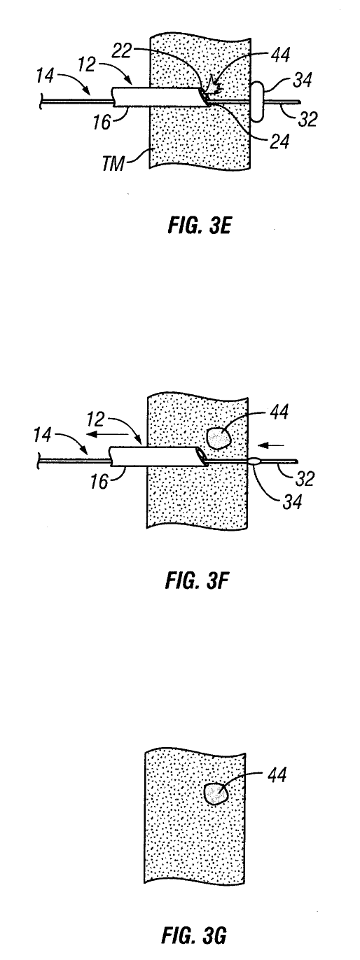

In the operational example of FIGS. 3A-3G, the above described injection system 10 is used to inject a substance into a tissue mass TM. To the right of the tissue mass TM is an open or fluid-filled area, cavity or body lumen. As seen in FIG. 3A, the shaft 32 of the anchoring member 14 is advanced, with the balloon 34 deflated, through the tissue mass TM such that a distal portion of the shaft 32 including the balloon 34 is within the open or fluid-filled area, cavity or body lumen.

Thereafter, as seen in FIG. 3B, an inflation device (e.g., a syringe, pump, PCTA balloon inflator, etc.) is attached to the Luer hub 38 of the anchoring member 14 and inflation fluid is injected through the inflation lumen 42 of the anchoring member shaft 32 causing the balloon 34 to inflate.

Thereafter, as seen in FIG. 3C, the anchoring member 14 is retracted (i.e., pulled in the proximal direction) until resistance is felt indicating that the proximal surface of the inflated balloon 34 has come into abutment with the wall of the open or fluid-filled area, cavity or body lumen adjacent to the tissue mass TM.

Thereafter, as seen in FIG. 3D, the injection cannula 12 is advanced into the tissue mass TM until the graduated distance markers 40 indicate that the outlet opening at the distal end of the infusion lumen 22 is a desired distance from the open or fluid-filled area, cavity or body lumen (i.e., a desired distance from the proximal surface of the inflated balloon 34, which is in abutment with the adjacent wall of the open or fluid-filled area, cavity or body lumen. In some applications of the method, the desired distance between the outlet opening at the distal end of the infusion lumen 22 and the open or fluid-filled area, cavity or body lumen may be determined on the basis of a pre-procedure imaging study wherein the thickness of the tissue mass TM has been determined. For example, in a case where the tissue mass TM consists of the ventricular wall of the myocardium and the open or fluid-filled area, cavity or body lumen consists of the adjacent ventricular chamber of the heart, a pre-procedure imaging study (e.g., an echocardiogram, high speed CT scan or other imaging technique) may be performed to obtain a measurement of the ventricular wall thickness at the location where the substance is to be injected. If, for example, the ventricular wall thickness is determined to be 20 millimeters and it is desired to deposit the substance in the transverse center of the ventricular wall, the injection cannula 12 will be advanced to a position where the fourth marker 40 is flush with the proximal end of the hub 26, the distance between the distal opening of the infusion lumen 22 and the proximal side of the balloon 34 (when inflated) is 10 millimeters.

Thereafter, with the injection cannula 12 at the desired position, an injector (e.g., a syringe, pump or other infusion apparatus) attached to the side Luer port 30 and is used to inject the desired substance 44 through injection lumen 22, as seen in FIG. 3E.

As shown in FIG. 3F, after the substance 44 has been injected, the balloon 34 is deflated and the injection cannula 12 and anchoring member 14 are removed.

Thus, the procedure is useable to deposit a quantity of the substance 44 at a desired depth or location within the tissue mass TM, as seen in FIG. 3G. The desired depth or location may be specifically selected to be within an area of target tissue (e.g., an infarct zone or area or ischemia) and to avoid or minimize any potential for inadvertently injecting the substance 44 into the adjacent hollow or fluid-filled open area, cavity or lumen.

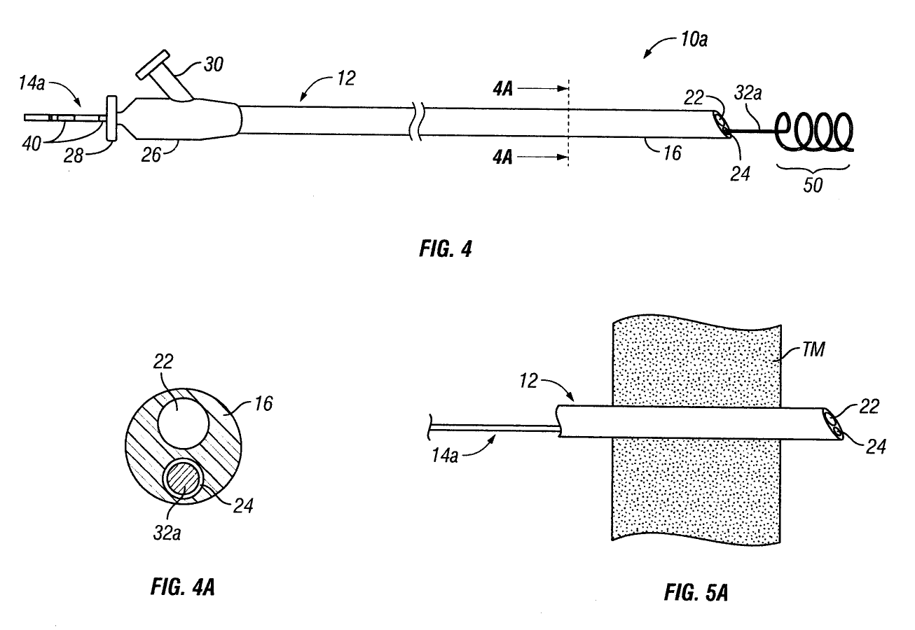

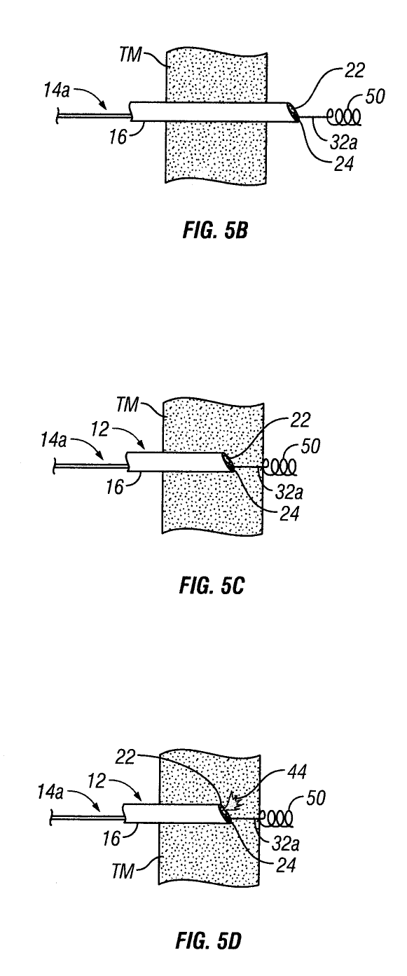

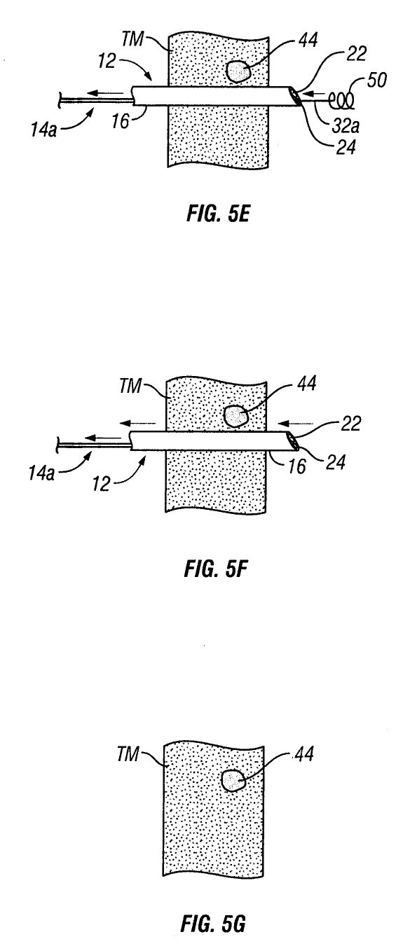

FIGS. 4 through 5G show another embodiment of an injector system 10a of the present invention. In this example, the system comprises the same injection cannula 12 as described above in combination with a different anchoring member 14a. This coiling-type anchoring member 14a shares numerous components with the balloon-type anchoring member 14 described above, including the straight proximal Luer hub 38 and the graduated distance markers 40. However, in this embodiment of the anchoring member 14a, the shaft 32a is devoid of any inflatable balloon and, instead, incorporates a coiling distal shaft segment 50. This coiling distal shaft segment 50 is formed of elastic or superelastic material (e.g., wire formed of nickel-titanium alloy (Nitinol)) that is biased to a coiled configuration (as shown) but is sufficiently resilient to assume a substantially linearized configuration when retracted into the anchoring member lumen 24 of the injection cannula 12. In the particular example of FIGS. 4A-5G, this anchoring member 14a is used in conjunction with the same dual lumen injection cannula 12 described above. As shown, the coiling-type anchoring member 14a extends through anchoring member lumen 24. Initially, the coiling distal shaft segment 50 is in a retracted position wherein it is disposed in a generally linear configuration within the anchoring member lumen 24. When desired, the operator may advance the coiling distal shaft segment 50 out of the distal end opening of anchoring member lumen 24, thereby freeing the coiling distal shaft segment 50 from surrounding constraint and allowing it to self expand to the helically coiled configuration seen in FIG. 4.

In the operational example of FIGS. 5A-5G, this embodiment of the injection system 10a is used to inject a substance into a tissue mass TM. To the right of the tissue mass TM is an open or fluid-filled area, cavity or body lumen. As seen in FIG. 5A, while the anchoring member 14a is in a retracted position with its coiling shaft segment 50 in its substantially linearized configuration within anchoring member lumen 24, the injection cannula is advanced fully through the tissue mass to a position where the distal end of the injection cannula 12 has emerged out of the tissue mass TM and into the adjacent open or fluid-filled area, cavity or body lumen.

Thereafter, as seen in FIG. 5B, the operator will advance the anchoring member shaft 32a to cause the coiling distal shaft segment 50 to emerge out of the distal end opening of anchoring member lumen 24, allowing the coiling distal shaft segment 50 self-expand to the helically coiled configuration.

Thereafter, as seen in FIG. 5C, the injection cannula 12 is retracted (i.e., pulled in the proximal direction) back into the tissue mass TM and the anchoring member 14a is retracted (i.e., pulled in the proximal direction) until resistance is felt indicating that the coiling distal shaft segment 50 has come into abutment with the wall of the open or fluid-filled area, cavity or body lumen adjacent to the tissue mass TM.

Thereafter, as seen in FIG. 5D, the position of the injection cannula 12 may be adjusted as necessary within the tissue mass TM until the graduated distance markers 40 indicate that the outlet opening at the distal end of the infusion lumen 22 is a desired distance from the open or fluid-filled area, cavity or body lumen (i.e., a desired distance from the proximal end of the coiled distal shaft segment 50 which is in abutment with the adjacent wall of the open or fluid-filled area, cavity or body lumen). As described above with regard to the first embodiment 10, in some applications of this method, the desired distance between the outlet opening at the distal end of the infusion lumen 22 and the open or fluid-filled area, cavity or body lumen may be determined on the basis of a pre-procedure imaging study wherein the thickness of the tissue mass TM has been determined. For example, in a case where the tissue mass TM consists of the ventricular wall of the myocardium and the open or fluid-filled area, cavity or body lumen consists of the adjacent ventricular chamber of the heart, a pre-procedure imaging study (e.g., an echocardiogram, high speed CT scan or other imaging technique) may be performed to obtain a measurement of the ventricular wall thickness at the location where the substance is to be injected. If, for example, the ventricular wall thickness is determined to be 20 millimeters and it is desired to deposit the substance in the transverse center of the ventricular wall, the injection cannula 12 will be advanced to a position where the fourth marker 40 is flush with the proximal end of the hub 26, the distance between the distal opening of the infusion lumen 22 and the proximal end of the coiled distal shaft segment 50 is 10 millimeters. With the injection cannula 12 having been maneuvered to its desired position, an injector (e.g., a syringe, pump or other infusion apparatus) attached to the side Luer port 30 and is then used to inject a desired substance 44 through injection lumen 22, as shown.

Thereafter, as seen in FIG. 5E, after the desired quantity of substance 44 has been injected, the injection cannula 12 and anchoring member 14a are advanced in the distal direction until the distal end of the injection cannula 12 has one again emerged out of the tissue mass TM and into the adjacent open or fluid-filled area, cavity or body lumen.

Then, as shown in FIG. 5F, the anchoring member 14a is retracted to a retracted position wherein its coiling shaft segment 50 is once again disposed in its substantially linearized configuration within anchoring member lumen 24 and the injection cannula 12 and anchoring member 14a are removed.

Thus, the procedure is useable to deposit a quantity of the substance 44 at a desired depth or location within the tissue mass TM, as seen in FIG. 5G. The desired depth or location may be specifically selected to be within an area of target tissue (e.g., an infarct zone or area or ischemia) and to avoid or minimize any potential for inadvertently injecting the substance 44 into the adjacent hollow or fluid-filled open area, cavity or lumen.

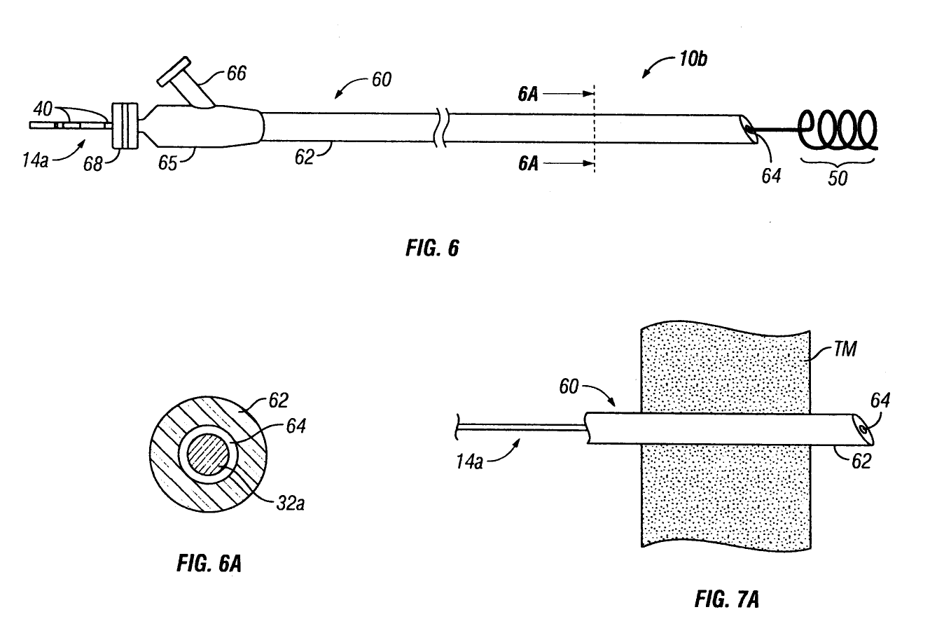

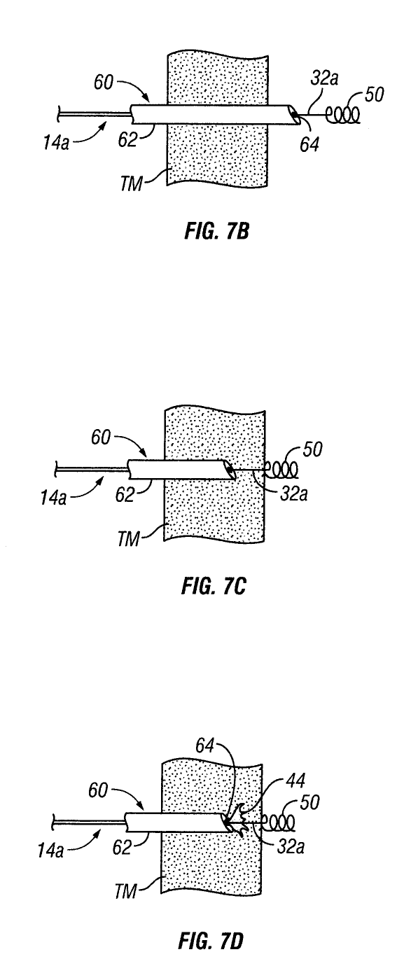

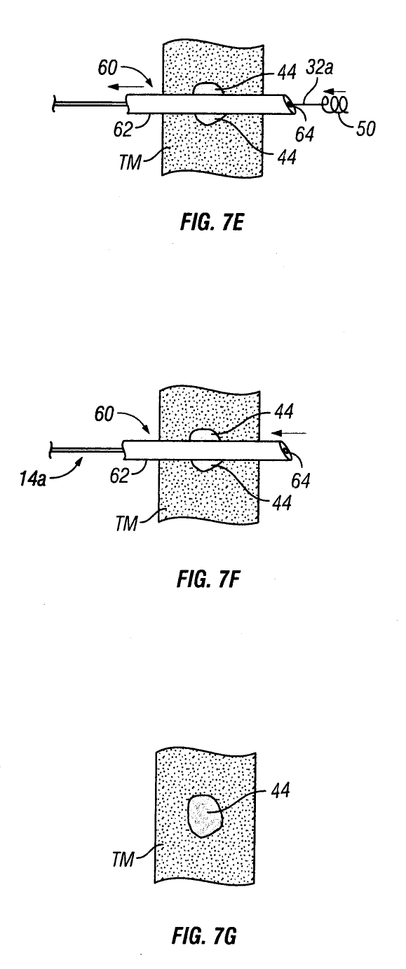

FIGS. 6 through 7G show another embodiment of an injector system 10b of the present invention. In this example, the system 10b comprises injection cannula 60 that has a combined infusion/anchoring member lumen 64 in combination with the coiling-type anchoring member 14b. This coiling-type anchoring member 14a is the same as that is shown in FIG. 4 and described above. It is to be appreciated, however, that as an alternative the balloon-type anchoring member 14 described above (or any other suitable anchoring member) could be used in place of this coiling-type anchoring member 14a. The injection cannula 60 used in this example comprises an elongate cannula shaft 62 having an infusion/anchoring member lumen 64 extending longitudinally therethrough. Additional other lumens may also be provided in some variations of this injection cannula 60. A proximal hub 65 is attached to the proximal end of the elongate cannula shaft 62. The proximal hub 65 has a proximal anchoring member port 68 which may include a Touhy-Borst valve or other apparatus that seals around the anchoring member shaft 32a when it is inserted through the proximal anchoring member port 68 to deter fluid from backflowing out of the proximal anchoring member port 68. The proximal anchoring member port 68 communicates with the infusion/anchoring member lumen 64. A side Luer port 66 also communicates with the infusion/anchoring member lumen 64. The anchoring member 14a is positioned within the infusion/anchoring member lumen 24 with its proximal end extending out of the proximal anchoring member port 68, as shown. Initially, the coiling distal shaft segment 50 of the anchoring member 14a is in a retracted position wherein it assumes a generally linear configuration within the infusion/anchoring member lumen 64. When desired, the operator may advance the coiling distal shaft segment 50 out of the distal end opening of infusion/anchoring member lumen 64, thereby freeing the coiling distal shaft segment 50 from surrounding constraint and allowing it to expand to the helically coiled configuration seen in FIG. 6. As may be appreciated from the cross sectional showing of FIG. 6A, in this embodiment, the diameter of the infusion/anchoring member lumen 64 is preferably larger than the outer diameter of the anchoring member shaft 32a so that a fluid may flow through the lumen 64 around the anchoring member shaft 32a. When it is desired to inject a substance, the Touhy-Borst valve or other closure apparatus on the proximal anchoring member port 68 will be tightened or otherwise caused to form a substantially fluid-tight seal around the anchoring member shaft 32a. The substance will then be injected through side Luer port 66 such that the substance will flow in the distal direction through infusion/anchoring member lumen 64, around the anchoring member shaft 32a, and out of the open distal end of the infusion/anchoring member lumen 64.

In the operational example of FIGS. 7A-7G, this embodiment of the injection system 10b is used to inject a substance into a tissue mass TM. To the right of the tissue mass TM is an open or fluid-filled area, cavity or body lumen. As seen in FIG. 7A, while the anchoring member 14a is in a retracted position with its coiling shaft segment 50 in its substantially linearized configuration within infusion/anchoring member lumen 64, the injection cannula 60 is advanced fully through the tissue mass TM to a position where the distal end of the injection cannula shaft 62 has emerged out of the tissue mass TM and into the adjacent open or fluid-filled area, cavity or body lumen.

Thereafter, as seen in FIG. 7B, the operator will advance the anchoring member shaft 32a to cause the coiling distal shaft segment 50 to emerge out of the distal end opening of infusion/anchoring member lumen 64, allowing the coiling distal shaft segment 50 self-expand to the helically coiled configuration.

Thereafter, as seen in FIG. 7C, the injection cannula 60 is retracted (i.e., pulled in the proximal direction) back into the tissue mass TM and the anchoring member 14a is retracted (i.e., pulled in the proximal direction) until resistance is felt indicating that the coiling distal shaft segment 50 has come into abutment with the wall of the open or fluid-filled area, cavity or body lumen adjacent to the tissue mass TM.

Thereafter, as seen in FIG. 7D, the position of the injection cannula 60 may be adjusted as necessary within the tissue mass TM until the graduated distance markers 40 on the anchoring member shaft 32a indicate that the outlet opening at the distal end of the infusion/anchoring member lumen 64 is a desired distance from the open or fluid-filled area, cavity or body lumen (i.e., a desired distance from the proximal end of the coiled distal shaft segment 50 which is in abutment with the adjacent wall of the open or fluid-filled area, cavity or body lumen). As described above, in some applications of this method, the desired distance between the outlet opening at the distal end of the infusion/anchoring member lumen 64 and the open or fluid-filled area, cavity or body lumen may be determined on the basis of a pre-procedure imaging study wherein the thickness of the tissue mass TM has been determined. For example, in a case where the tissue mass TM consists of the ventricular wall of the myocardium and the open or fluid-filled area, cavity or body lumen consists of the adjacent ventricular chamber of the heart, a pre-procedure imaging study (e.g., an echocardiogram, high speed CT scan or other imaging technique) may be performed to obtain a measurement of the ventricular wall thickness at the location where the substance is to be injected. If, for example, the ventricular wall thickness is determined to be 20 millimeters and it is desired to deposit the substance in the transverse center of the ventricular wall, the injection cannula 60 will be advanced to a position where the fourth marker 40 is flush with the proximal end of the hub 65, the distance between the distal opening of the infusion/anchoring member lumen 64 and the proximal end of the coiled distal shaft segment 50 is 10 millimeters. After the injection cannula 60 has been 12 having been placed in the desired position, an injector (e.g., a syringe, pump or other infusion apparatus) attached to the side Luer port 66, the Touhy-Borst valve or other closure apparatus on proximal anchoring member port 68 is tightened or otherwise caused to form a substantially fluid-tight seal around the anchoring member shaft 32a and the injector is then used to inject the substance 44 through side Luer port 66 such that the substance 44 flows in the distal direction through infusion/anchoring member lumen 64, around the anchoring member shaft 32a, and out of the open distal end of the infusion/anchoring member lumen 64, as shown.

Thereafter, as seen in FIG. 7E, after the desired quantity of substance 44 has been injected, the injection cannula 60 and anchoring member 14a are advanced in the distal direction until the distal end of the injection cannula 12 once again emerges out of the tissue mass TM and into the adjacent open or fluid-filled area, cavity or body lumen.

Then, as shown in FIG. 7F, the anchoring member 14a is retracted to a retracted position wherein its coiling shaft segment 50 is once again disposed in its substantially linearized configuration within the infusion/anchoring member lumen 64 and the injection cannula 60 and anchoring member 14a are removed.

Thus, the procedure is useable to deposit a quantity of the substance 44 at a desired depth or location within the tissue mass TM, as seen in FIG. 7G. The desired depth or location may be specifically selected to be within an area of target tissue (e.g., an infarct zone or area or ischemia) and to avoid or minimize any potential for inadvertently injecting the substance 44 into the adjacent hollow or fluid-filled open area, cavity or lumen.

In some applications of this invention, the injection cannula 12, 60 may be directly inserted, either percutaneously or via an incision formed in a body wall, and advanced into a desired organ or body structure. For example, if it is desired to deliver a substance or device into the myocardium of the heart, in some embodiments, the injection cannula 12, 60 may be inserted through an open thoracotomy incision or through a thoracoscopic port and then advanced through the epicardial surface of the heart and to a desired position at a predetermined depth within the myocardium in accordance with any of the above-described procedures. In other applications, the injection cannula 12, 60 may comprise a straight or curved cannula that is advanceable from a transluminally inserted tissue penetrating catheter. In such cases, the tissue penetrating catheter will be initially inserted into the subject's vasculature and advanced to a position within a blood vessel near or within the anatomical structure in which it is intended to deliver the substance or device. Thereafter, the injection cannula 12, 60 and accompanying anchoring member 14, 14a will be advanced from the catheter and used to effect the intended delivery of the substance or device in accordance with the procedures described herein. Examples and details of tissue penetrating catheters that may be modified to incorporate the systems 10, 10a, 10b of this invention include but are not limited to those described in U.S. Pat. No. 5,830,222 (Makower); U.S. Pat. No. 6,068,638 (Makower), U.S. Pat. No. 6,159,225 (Makower), U.S. Pat. No. 6,190,353 (Makower, et al.), U.S. Pat. No. 6,283,951 (Flaherty, et al.), U.S. Pat. No. 6,375,615 (Flaherty, et al.), U.S. Pat. No. 6,508,824 (Flaherty, et al.), U.S. Pat. No. 6,544,230 (Flaherty, et al.), U.S. Pat. No. 6,655,386 (Makower et al.), U.S. Pat. No. 6,579,311 (Makower), U.S. Pat. No. 6,602,241 (Makower, et al.), U.S. Pat. No. 6,655,386 (Makower, et al.), U.S. Pat. No. 6,660,024 (Flaherty, et al.), U.S. Pat. No. 6,685,648 (Flaherty, et al.), U.S. Pat. No. 6,709,444 (Makower), U.S. Pat. No. 6,726,677 (Flaherty, et al.) and U.S. Pat. No. 6,746,464 (Makower) and co-pending U.S. patent applications having Ser. Nos. 11/279,993; 11/279,265; 11/279,771; 11/610,092; 11/534,895; 11/613,764; 11/837,718; 12/054,533 and 12/045,120, the entire disclosure of each such patent and patent application being expressly incorporated herein by reference. Also, there exists a commercially available tissue penetrating catheter of this type which includes an on-board ultrasound imaging transducer in combination with a marker that provides an image of the target location along with an indication of the projected penetrator trajectory relative to the target location (i.e., the Pioneer™ Catheter, Medtronic Vascular, Inc., Santa Rosa, Calif.).

In some embodiments where the systems and methods of the present invention are used to deliver a therapeutic substance to a desired location (e.g., an ischemic or infracted region) within the myocardium of a subject's heart, the types of therapeutic substances that may be injected include but are not limited to angiogenic substances, cell preparations or other substances establish new functional cardiac muscle cells, deter ventricular remodeling, deter scar formation at an infarct site or otherwise improve/maintain myocardial function. In some instances, these therapeutic substances may be combined with other ancillary agents (e.g., potentiators or radiographic markers) when injected into the myocardium. Examples of angiogenic substances that may be delivered using the systems and methods of the present invention include peptide growth factors, small molecule drugs and other active compounds, biologically active carbohydrates, recombinant biopharmaceuticals, agents that are active in the regulation of vascular physiology, cellular and gene therapy agents, nitric oxide, agents that affect transcription or turnover of cellular mRNA or the efficiency with which specific mRNA translates into its protein product, antisense agents, hormones, soluble receptors, receptor ligands, synthetic and naturally occurring peptides, peptidomimetic compounds, specific and non-specific protease inhibitors, postaglandins, inhibitors of prostaglandin synthase and/or other enzymes involved in the regulation of prostaglandin synthesis, fibroblast growth factors (FGF's), acidic (aFGF, FGF-II) and basic (bFGF, FGF-I) fibroblast growth factors, vascular endothelial growth factors (VEGF), agents that stimulate endogenous production of VEGF such as platelet-derived growth factor (PDGF), purified monocyte-derived angiogenic substance (angiotropin), angiogenin, transforming growth factor alpha (TGF-α), transforming growth factor beta (TGF-β) and angiogenic cell precursors (e.g., stem cells). Examples cell preparations that may be delivered using the systems and methods of the present invention for the purpose of establishing new functional heart muscle include; myocyte preparations, pluripotent cells (e.g., embryonic, adult and mesenchymal stem cell preparations), myoblast (including in some cases skeletal myoblast) preparations, bone marrow mononuclear cell preparations, etc. Also, the systems and methods of the present invention may be used to deliver platelet gel through a single infusion lumen or components of platelet gel (e.g., platelet concentrate and a thrombin containing solution) through separate lumens of the injection cannula 12, 60 so that they become combined to form platelet gel immediately before or after entering the myocardium.

Although specific reference has been made to use of the invention to deliver substances or implantable devices into the myocardium adjacent to a chamber of the heart, it is to be appreciated that the invention may be used to deliver substances or devices to various other normal or aberrant tissue masses located adjacent to other open or fluid-filled spaces, cavities or lumens of a subject's body. For example, the systems and methods of the present invention may be used to deliver various diagnostic, cosmetic or therapeutic substances (drugs, chemotherapeutic agents, other anti-cancer agents, radiographic contrast materials, biologics, cell preparations, gene therapy preparations, bulking agents, fillers, etc.) or to deliver implantable devices (e.g., drug delivery implants, electrodes, radiographic markers or tags, nanotechnology devices, etc.) through needles, cannulae or other elongate devices that have been positioned, in accordance with the present invention, at specific desired locations within organs or tissue masses (normal or aberrant) within the bodies of human or animal subjects.

For example, the systems and methods of the present invention may be used to deliver a substance or device into brain tissue at a predetermined distance from a ventricle of the brain. Also, in subjects who suffer from certain bladder cancers, the systems and methods of the present invention may be used to deliver substances (e.g., cancer treatment agents) or devices (e.g., drug delivery or radiotherapy implants) into tumor tissue at a predetermined distance from the interior cavity of the urinary bladder. The systems and methods of the present invention may also be used for delivery of a substance or device into tissue of the eye (e.g. corneal or scleral tissue) at a predetermined distance from an intraocular space such as the anterior chamber of the eye.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A system for positioning a treatment delivering location of a treatment device at a desired location within a tissue mass located near an open or fluid-filled space, body cavity or body lumen, said system comprising:
a treatment device having a treatment delivery location that is advanceable into the tissue mass;
an anchoring member comprising a shaft and an anchor, the anchor having a radially collapsed configuration and a radially expanded configuration, the shaft being advanceable into the space, body cavity or body lumen while the anchor is in its collapsed configuration, the anchor being thereafter transitionable to an expanded configuration and the shaft being thereafter retractable to pull the anchor into abutment with a wall of the space, body cavity or body lumen; and
measuring apparatus useable to determine the distance between the location at which the anchor abuts against the wall of the space, body cavity or body lumen and a treatment-delivering location on the elongate treatment device.

2. A system according to claim 1 wherein the treatment device comprises an elongate member having a lumen through which the shaft of the anchoring member advances and retracts.

3. A system according to claim 2 wherein the treatment delivery location comprises port for infusing a substance through the same lumen through which the shaft of the anchoring member advances and retracts.

4. A system according to claim 3 wherein the treatment device has an elongate shaft and a proximal hub that includes an anchoring member port through which the anchoring member shaft extends into the lumen and an infusion port through which a substance may be infused through the lumen.

5. A system according to claim 4 wherein the treatment device further comprises an apparatus which forms a substantially fluid tight seal about the shaft of the anchoring member to deter a substance from backflowing out of the anchoring member port when said substance is being infused through the lumen.

6. A system according to claim 2 wherein the treatment device further comprises a at least one delivery lumen that is separate from the lumen through which the shaft of the anchoring member advances.

7. A system according to claim 6 wherein the treatment device has a proximal hub that includes an anchoring member port through which the anchoring member shaft extends into the through which the shaft of the anchoring member advances and retracts and a treatment delivery port through which a substance or device may be delivered through the delivery lumen.

8. A system according to claim 1 wherein the anchor comprises a balloon.

9. A system according to claim 1 wherein the anchor comprises a coiling member.

10. A system according to claim 1 wherein the measuring apparatus comprises graduated distance markings on the shaft of the anchoring member.

11. A method for positioning a treatment delivery location of a treatment device within a tissue mass at a desired distance from an open or fluid-filled space, body cavity or body lumen, said method comprising the steps of:
(A) advancing an anchoring member from the treatment device and into the space, body cavity or body lumen;
(B) causing an expandable anchor on the anchoring member to expand;
(C) retracting the anchoring member to cause the expanded anchor to abut against a wall of the space, body cavity or body lumen;
(D) measuring the distance between the treatment delivery location on the elongate treatment device and the location at which the anchor abuts against the wall of the space, body cavity or body lumen; and
(E) maneuvering the elongate treatment device as needed until the measured distance indicates that the treatment delivery location of the elongate treatment device is positioned at the desired distance from the space, body cavity or body lumen.

12. A method according to claim 11 further comprising the step of:
delivering a treatment from the treatment delivery location into the tissue mass.

13. A method according to claim 12 wherein the treatment delivery location comprises and outlet opening and wherein the step of delivering a treatment from the treatment delivery location into the tissue mass comprises delivering a substance or implantable device out of the outlet opening and into the tissue mass.

14. A method according to claim 11 wherein the tissue mass comprises the myocardium of a subject's heart and the space, body cavity or body lumen comprises a chamber of the heart.

15. A method according to claim 11 wherein the tissue mass comprises brain tissue and the space, body cavity or body lumen comprises a ventricle of the brain.

16. A method according to claim 11 wherein the tissue mass comprises a urinary bladder tumor or mass and the space, body cavity or body lumen comprises the urinary bladder.

17. A method according to claim 11 wherein the expandable anchor comprises a balloon and wherein Step B comprises inflating the balloon.

18. A method according to claim 11 wherein the expandable anchor comprises a coiling member and wherein Step B comprises causing the coiling member to become coiled.

19. A method according to claim 11 wherein the anchoring member comprises an elongate anchoring member shaft with said anchor formed or attached thereto with graduated distance markings on the anchoring member shaft, said graduated distance markings being used in Step D to measure the distance between the treatment delivery location on the elongate treatment device and the location at which the anchor abuts against the wall of the space, body cavity or body lumen.

20. A method according to claim 12 wherein the treatment delivery location comprises at least one outlet opening through which at least one substance or device is expelled out of the elongate treatment device and wherein the step of delivering a treatment from the treatment delivery location into the tissue mass comprises expelling a substance or device out of said opening.

22. A method according to claim 20 wherein i) the tissue mass comprises an ischemic or infracted area of the myocardium of a subject's heart, ii) the space, body cavity or body lumen comprises a chamber of the heart and iii) the step of delivering a treatment from the treatment delivery location into the tissue mass comprises expelling a therapeutic substance into the ischemic or infracted area of the myocardium.

23. A method according to claim 22 wherein the therapeutic substance comprises an angiogenic agent.

24. A method according to claim 23 wherein the angiogenic agent comprises at least one substance selected from the group consisting of: peptide growth factors, small molecule drugs and other active compounds, biologically active carbohydrates, recombinant biopharmaceuticals, agents that are active in the regulation of vascular physiology, cellular and gene therapy agents, nitric oxide, agents that affect transcription or turnover of cellular mRNA or the efficiency with which specific mRNA translates into its protein product, antisense agents, hormones, soluble receptors, receptor ligands, synthetic and naturally occurring peptides, peptidomimetic compounds, specific and non-specific protease inhibitors, postaglandins, inhibitors of prostaglandin synthase and/or other enzymes involved in the regulation of prostaglandin synthesis, fibroblast growth factors (FGF's), acidic fibroblast growth factors (aFGF, FGF-II), basic fibroblast growth factors (bFGF, FGF-I), vascular endothelial growth factors (VEGF), agents that stimulate endogenous production of VEGF, platelet-derived growth factor (PDGF), purified monocyte-derived angiogenic substance (angiotropin), angiogenin, transforming growth factor alpha (TGF-α), transforming growth factor beta (TGF-β) and angiogenic cell precursors.

25. A method according to claim 22 wherein the therapeutic substance comprises a cell preparation or substance that establishes new heart muscle.

26. A method according to claim 25 wherein the cell preparation or substance that establishes new heart muscle comprises at least one substance selected from the group consisting of: myocytes, pluripotent cells; embryonic stem cells, adult stem cells, mesenchymal stem cells, myoblasts, skeletal myoblasts and bone marrow mononuclear cells.

27. A method according to claim 22 wherein the therapeutic substance comprises a substance that deters ventricular remodeling or improves myocardial function.

28. A method according to claim 27 wherein the substance comprises platelet gel.

29. A method according to claim 28 wherein the platelet gel is injected through a lumen of the treatment device.

30. A method according to claim 28 wherein components of platelet gel are injected through separate lumens of the treatment device so that said components of platelet gel become combined to form platelet gel immediately before or after entering the myocardium.
